(19) United States
(12) Patent Application Publication
(10) Pub. No.: US 2016/0250378 A1
(43) Pub. Date: Sep. 1, 2016

(54) Spray-On Burn Dressing

(71) Applicant: ADVANCED FIRST AID RESEARCH PTE. LTD., Singapore (SG)

(72) Inventors: Carl Freer, Singapore (SG); Joseph Marten, Singapore (SG); Stephen Carroll, Singapore (SG); Zhilong Shi, Singapore (SG)

(73) Assignee: Advanced First Aid Research PTE. LTD., Singapore (SG)

(21) Appl. No.: 15/030,512

(22) PCT Filed: Oct. 20, 2014
(86) PCT No.: PCT/SG2014/000494

§ 371 Date: Apr. 19, 2016

Related U.S. Application Data
(60) Provisional application No. 61/893,355, filed on Oct. 21, 2013.

(51) Int. Cl. A61L 26/00

(52) U.S. Cl. CPC A61L 26/0076; A61L 26/0004; A61L 26/0023; A61L 26/0014; A61L 26/0066; A61L 2400/12; A61L 2300/40; A61L 2300/236

(57) ABSTRACT

The invention is a class of spray-on burn dressings for use in the treatment of various types of tissue burns, such as burns due to heat, chemicals, or sun exposure. The inventive spray-on dressing is comprised of a film-forming polymer and metal or metal oxide particles. The metal or metal oxide, preferably aluminum oxide, acts as a thermally conductive component to enhance heat-dissipation from the burn. The spray-on dressing may also include antibacterial agents, anesthetics, analgesics, or preservatives. The spray-on dressing may be dispensed by aerosol container or by manual pump spray.

### BACKGROUND OF THE INVENTION

This PCT application claims the benefit under 35 U.S.C. § 119(e) of U.S. provisional patent application Ser. No. 61/893,355, entitled “Spray-On Burn Dressing” filed on Oct. 21, 2013, the entire disclosure of which is incorporated herein by reference.

Burn injuries are caused by fire, chemicals, electricity, and friction and can vary in severity. First degree burns are the least severe, causing redness, and healing relatively quickly. On the other end of the spectrum, fourth degree burns are the most severe, burning down to the level of the muscle and bone. Second and third degree burns fall between these extremes.

Medical professionals often try to strike a balance when deciding how to treat burns. On one hand, if a burn is superficial and relatively dry, then it may be desirable to keep the wound moist with water or some sort of ointment or cream. However, a problem with applying many ointments and/or creams is that such applications often do not help draw heat away from a wound. On the other hand, if a burn is more serious, such as a second-degree burn that is oozing fluid, then there is an enhanced fear of infection. In such cases, some medical professionals feel that such wounds should be kept relatively dry, while still others may advocate for the application of various ointment dressings with antibiotic properties to fight infection. Hence, it would be desirable to come up with a treatment strategy that is able to provide the best of all worlds.

On Aug. 30, 1948, Time Magazine reported that steam from an exploding locomotive had scalded Fireman Frank Mihlan of the Erie Railroad. When Mihlan was carried into Cleveland's Charity Hospital on Jul. 15, 1948, 70% of his body was burned, and doctors thought that Mihlan had little chance of survival. However, attending surgeons decided to try wrapping the Mihlan's burns in thin strips of aluminum foil, a technique developed by Toronto's Dr. Alfred W. Farmer. It was the first time that aluminum foil for burns had been used in the U.S.; the first time it had ever been used for burns of the whole body. Relief from pain was “miraculous”, and within 20 minutes of application, Mihlan was resting comfortably. As an added precaution, Mihlan was given intravenous fluids and penicillin. The aluminum foil, which looked like the inside wrapping of a cigarette package, apparently acted as a seal for the body fluids that seep from burned surfaces. It also apparently helped kill bacteria, speeding the healing process. Twelve days after being bandaged in the aluminum foil wrappings, Mihlan was out of bed. Eventually, Mihlan left the hospital unscarred, albeit temporarily reddened.

Bandages and wraps may incorporate a thin layer of thermally conductive metal (such as aluminum) at the base of a substrate adapted to be in direct contact with a burn wound, while the top side of the aluminum substrate has a heat-dissipation-enhancing topography to help cool burns faster by enhancing thermal convection properties. Such products are described in Aluminaid's U.S. Pat. No. 8,530,720 to Freer, et al.

Spray-on wound dressings are convenient, easy ways to protect wounds during the healing process and avoid certain drawbacks of conventional dressings such as physical mobility, pain, and peeling of hair during removal. Currently there is no burn dressing product available to the general public that employs medical advantages of aluminum (or other metals) in a convenient spray-on dressing.

It would be advantageous to develop aluminum or other metal-infused healing/therapeutic dressing products that are easy for a consumer to safely use in a convenient, aerosol or manual-pump spray.

### SUMMARY OF THE INVENTION

The invention is a class of medical products, more particularly a spray-on burn dressing containing a metal or metal oxide. The burn dressings are designed to alleviate discomfort and relieve pain caused by burns. The metal or metal-oxide, and in particular aluminum oxide (Al₂O₃), is included as a component of the dressing for its effective heat-transfer and wound healing characteristics. The inventive dressing provides a metalized mass at the burn site to enhance heat-dissipation and to assist in the healing of the burn.

The metal or metal oxide is dispersed within a liquid or gel comprising a film forming polymer to form the base composition. In one embodiment aluminum is selected as the metal. In one embodiment aluminum oxide is selected as the metal oxide. Microparticles (diameter larger than about 0.1 micrometers and preferably between about 5 to about 100 micrometers) or nanoparticles (diameter smaller than about 100 nanometers) of metal or metal oxide are dispersed in the film-forming polymer. Solvents such as alcohols, ethyl acetate, or water may be used to disperse the metal or metal oxide in the polymer and to dissolve the polymer. Surface modification of metal or metal oxide particles may be used to increase particles' stability and compatibility in the solvents. In-situ or emulsion polymerization of one monomer with particles may be used to disperse the particles. In-situ or miniemulsion copolymerization of one monomer with hydrophilic groups and one monomer with hydrophobic groups with metal or metal oxide particles may also be used to disperse the metal or metal oxide particles in as-prepared copolymer. Surface-initiated polymerization may also be used to make the particle-dispersed polymer suspension. Viscosity may be controlled by adjusting the concentration of polymer and metal or metal oxide to attain preferred characteristics.

Another aspect of the invention is directed to the method of applying the spray-on burn dressing. In one embodiment the burn dressing is applied using an aerosol spray. In another embodiment the burn dressing is applied with a manual pump spray. In each embodiment, the method enhances heat-dissipation to assist in the healing of a burn. It is preferred that the spray-on burn dressing has a drying time of less than about a minute, and more preferably less than about 30 seconds. It is preferred that the spray-on burn dressing remain flexible when dried to enhance patient comfort.

Various additional agents may be added to the base composition including: surfactants to modify metal or metal oxide surface properties or to enhance stability of the composition; antibacterial agents to suppress bacterial growth and assist with wound healing; anesthetics and analgesics to reduce pain; preservatives to increase shelf life of the composition; or anticorrosion agents to reduce or eliminate corrosion of the spray container.

In one embodiment heparan sulfate is included as an anticoagulant and promoter of wound healing.

### DETAILED DESCRIPTION OF THE INVENTION

There are three ways in which thermal energy transfer can be described: Conduction; Convection; and Radiation. Conduction requires physical contact (similar to the flow of electricity in wire). Convection emanates from the movement of molecules (e.g., the way in which heated and cooled water or other fluid moves up and down). Radiation does not necessarily involve direct contact (e.g., the way the sun emits light rays).

At any given temperature, a given mass of aluminum holds much less energy than an equivalent mass of human flesh. For instance, in convection or conduction, if one touches aluminum foil from an oven during the cooking process, a subject's hand and the foil share the thermal energy. The hand (of much greater mass) requires much more energy to raise its temperature (if at all, depending upon the physical connection between the foil and the food). When the subject touches aluminum foil, the foil transfers heat to the flesh; however, due to the aluminum's low specific-heat capacity, the foil quickly loses energy, barely raising the temperature of the skin in contact. Because aluminum foil does not effectively store conducted heat it therefore facilitates the “cooling” of a burn.

While aluminum does not effectively store conducted heat, aluminum is nonetheless an excellent conductor of heat. Aluminum conducts heat away from the source and readily gives the heat up to its surroundings. This has a cooling effect to the source of the heat. Aluminum can be an effective conductor of a subject's body heat, alleviating pain which emanates from added warmth on a subject's burn.

The spray-on burn dressings of the invention contain a metal or metal oxide for enhanced heat-dissipation. The inventive dressings are designed to alleviate discomfort and pain caused by bums including those resulting from sun exposure, fire, chemicals, electricity, or friction. The metal or metal-oxide is included as a component of the dressing for its effective heat-transfer and wound healing characteristics. Because of the effective heat-transfer characteristics, the inventive dressings may also be used to alleviate pain cause by itching, inflammation, or rashes.

Various metals or metal oxides may be used in the inventive dressings and preferred metals or metal oxides are those with efficient heat-transfer qualities. Metals or metal oxides may also be chosen based on additional qualities such as toxicity, chemical reactivity, surface charge, or solvent compatibility. A particularly preferred metal oxide is aluminum oxide (Al₂O₃) because of its thermal conductivity.

Preferred thermally conductive metals include aluminum, silver, gold, copper, magnesium, tungsten, titanium, and platinum. Other preferred metals include iron, nickel, zinc, tin, and palladium. In one preferred embodiment the metal is aluminum. One metal or more than one metal may be used. In one embodiment both aluminum and silver microparticles are present in the dressing. Alloys substantially based on these metals and other biocompatible metal alloys may also be used. Such alloys include aluminum alloys, chromium/molybdenum/iron alloys, or aluminum/magnesium alloys. One preferred aluminum alloy contains at least about 90% aluminum. One preferred aluminum alloy contains at least 92% aluminum and about 5% magnesium. One metal alloy or more than one metal alloy may be used.

Preferred metal oxides include aluminum oxide, zinc oxide, silver oxide, copper oxide, magnesium oxide, titanium dioxide, tin oxide, and iron oxide. A particularly preferred metal oxide is aluminum oxide; another particularly preferred metal oxide is zinc oxide. One metal oxide or more than one metal oxide may be used. Another suitable oxide is silicon dioxide.

In one embodiment, the burn dressing includes one or more than one of a thermally conductive metal, metal alloy or metal oxide. In one embodiment the burn dressing includes aluminum metal particles and aluminum oxide particles to enhance thermal conductivity.

The metal or metal oxide utilized in the invention is preferably sized as microparticles (diameter larger than about 0.1 micrometers and preferably between about 5 to about 100 micrometers) or nanoparticles (diameter smaller than about 100 nanometers). The metal or metal oxide particles are preferably in the range from about 5 to about 100 micrometers. In one embodiment, the aluminum oxide particle diameters are less than about 100 nanometers. In another embodiment, the aluminum oxide particles are about 10 micrometers.

The metal or metal oxide is dispersed within a liquid or gel comprising a film forming polymer to form the base composition. Film forming polymers may be synthetic or natural. Suitable film-forming polymers may be sodium alginate, cellulose and cellulose derivatives, polyethylene glycol and its copolymer, polyacrylate and its copolymer, poly(methyl acrylate) and its copolymer, polyacrylamide and its copolymer, polyvinyl acetate and its copolymer, polyvinyl pyrrolidone and its copolymer, polyurethane and its copolymer, and polyisobutylene and its copolymer. Other suitable polymers include copolymers of monoalkyl esters of poly (methyl vinyl ether-co-maleic acid). Examples of suitable polymers may be obtained commercially from Ashland Inc. and are sold under the trade name Gantrez, including Gantrez polymers A-425, ES-225, ES-335, ES-425, ES-435, and SP-215. Other suitable polymers include poly(hexamethydisiloxane-co-acrylate-co-phenylmethylsiloxane), nitrocellulose, or ethyl cellulose. Still other suitable polymers include poly(ethoxyethyl methacrylate); poly(ethylhexyl acrylate-co-dimethylaminoethyl methacrylate-co-t-butylacrylamide); poly(2-ethylhexyl acrylate-co-butylacrylamide); poly(caprolactone) and its copolymer; gelatin; poly(di-lactide) and its copolymer; poly(oxyethylene-co-oxypropylene); gellan gum; xantham gum; and starch and its derivatives, and chitosan and its derivatives.

One film-forming polymer or more than one film-forming polymer may be used. Preferred film-forming polymers include polyacrylate copolymer (Avalure AC 120, Lubrizol), polyurethane copolymer (Avalure UR 450, Lubrizol), methyl vinyl ether/maleic acid copolymer (Gantrez, Ashland), PVM/MA copolymer (Omnirez 2000, Ashland), and cellulose derivatives.

Film-forming polymers may be selected based on the quality of film produced. Preferred films will protect the burn from environmental bacteria and dirt; preferred films will have moisture vapor permeability to prevent accumulation of aqueous fluid between the dressing and the burn; preferred films will be non-toxic, non-irritating, and do not burn or sting the skin when applied; preferred films should be readily removable when desired; preferred films will be uniform and will not crack. In one embodiment the film is water-proof; in one embodiment the film-forming polymer is an acrylate copolymer. In one embodiment the film is water washable; in one embodiment the film-forming polymer is selected from polyvinyl alcohol, polyvinyl pyrrolidone, polyvinyl acetate, or polyethylene glycol; in one embodiment the film-forming polymer is selected from hydroxypropyl cellulose, cellulose acetate butyrate, or alginate. In one embodiment the film adheres to skin and does not wash off with hot or cold water but can be washed off with soapy water. Film-forming polymers may also be selected based on the time required to form a film; high-viscosity polymers (used in the absence of solvent or with low amounts of solvent) may result in a solution having a high viscosity that may result in difficulty spraying.

Preferred films are tough, flexible and uniform without cracking, and exhibit tack-free adhesion. It is preferred that the spray-on dressing is tack-free in less than about a minute, and more preferably less than about 30 seconds.

It is a goal of the invention to ensure that the metal or metal oxide particles are evenly dispersed throughout the dressing and do not settle out. Solvents may be used to disperse the metal or metal oxide in the polymer and also to dissolve the polymer. Preferred solvents include water, methanol, ethanol, propanol, isopropanol, acetone, ethyl acetate, propylene glycol, glycerin, diethyl ether, and mixtures thereof. Preferred solvents include isopropanol, water, ethanol, ethyl acetate, and mixtures thereof.

One method of making the spray-on dressing comprises mixing the film-forming polymer with a solvent, and then adding metal oxide particles. The components are then stirred (with or without ultrasonication) until uniform at a temperature of about 20 to about 30 degrees Celsius. Unless otherwise stated herein, percentages are reported as weight/weight.

In one embodiment, 2.0 grams of Gantrez polymer methyl vinyl ether/maleic acid copolymer are combined with 50 ml of deionized water and 28 grams of isopropanol in a suitable vessel, and these components are mixed at about 40 to about 60 degrees Celsius until a clear solution is formed. Next 20 grams of aluminum oxide particles are added to the mixture and stirred at 20 to about 30 degrees Celsius until all particles are dispersed uniformly throughout the dressing.

One spray-on dressing comprises about 1% to about 20% film-forming polymer, about 10% to about 90% isopropanol, about 10% to about 40% water, and about 10% to about 30% metal oxide microparticles. One spray-on dressing comprises about 1% to about 3% film-forming polymer, about 40% to about 60% isopropanol, about 20% to about 30% water, and about 10% to about 30% metal oxide microparticles. One spray-on dressing comprises about 2% film-forming polymer, about 50% isopropanol, about 28% water, and about 20% metal oxide microparticles. In one embodiment, the spray-on dressing comprises about 5% poly(acrylate-co-octylacrylamide) copolymer, about 60% isopropanol, about 15% water, and about 20% aluminum oxide microparticles. In this embodiment the mean size of aluminum oxide microparticles is about 10 micrometers.

Viscosity of the dressing can be controlled by adjusting the concentration of polymer and metal or metal oxide. Surfactants such as Tween 80, Span 80 or Pluronic F-127 may be used to modify the metal or metal oxide surface properties to enhance stability and to enhance dispersion within the film-forming polymer.

One aspect of the invention is directed to the method of applying the burn dressing, and another aspect of the invention is directed to the method of treating a burn by applying the spray-on dressing to the burn. In one embodiment the burn dressing is applied using an aerosol spray. In another embodiment the burn dressing is applied with a manual pump spray. In a manual pump spray embodiment, the wound dressing composition is placed in a can or bottle operably connected with a manually operated spray. In an aerosol embodiment, the wound dressing composition is placed in a can or bottle along with a propellant under pressure and operably connected a valve (including an actuator, mounting cup, stem, stem gasket, spring, housing, and distube).

In an aerosol embodiment, suitable propellants include compressed gas (such as nitrogen, carbon dioxide, nitrous oxide, and air), hydrocarbons (such as propane, isobutane, butane and isopentane), dimethyl ether (DME) and fluorocarbons (such as HFC 134a and HFC 152a).

In an aerosol embodiment, the bottle's inner surface may be uncoated or coated, and coatings may include, for example, epoxy or polyimide. In an aerosol embodiment, the valve stem may be made of various materials including, for example, rubber.

In an aerosol embodiment, factors such as bottle material (metal, glass, plastic), dimensions, valve components, internal pressure and bottle inner coating may be varied depending on the propellant chosen as well as the final composition of the burn dressing.

In an aerosol embodiment containing isopropanol as a solvent, it is expected that some or all of the isopropanol may be eliminated if dimethyl ether is used as the aerosol propellant.

While it is preferable to apply the dressing to a burn using spray-on techniques, the dressing may also be applied to a burn with a tool such as a brush or spatula, may be applied using a roll-on applicator, or may be applied directly to the wound in bulk from a tube or packet.

In all cases, the burn dressing is applied to the area associated with a burn to enhance heat-dissipation from the burn to assist in the healing. In particular, the burn dressing conducts heat away from the burn and readily gives that heat up to the surrounding environment, which has a cooling effect.

Various additional agents may be added to the wound dressing composition including: antibacterial agents to suppress bacterial growth and assist with wound healing; anesthetics and analgesics to reduce pain; preservatives to increase shelf life of the composition; perfumes, dyes or pigments to enhance the look or fragrance of the dressing; or anti-corrosion agents to reduce or eliminate corrosion of the spray container. Each of these agents may be present in the burn dressing in an amount less than about 2.0%, less than about 1.0%, or less than about 0.5%.

Antibacterial agents may include metal ions (such as silver or copper ions) or metal salts (such as silver nitrate, lactate or citrate), metal nanoparticles (such as silver nanoparticles), antibacterial peptides, quaternary ammonium compounds, triclosan, iodine, PVP-iodine, phenol compounds, chlorhexidine gluconate, polyhexamide, silver sulfadiazine, octenidine, as well as antibiotics such as sulfate, beta-lactams, fluoroquinolones, aminoglycosides, glycopeptides, oxazolidinones, bacteriocin, or tetracycline. In a preferred embodiment silver ions are included in the burn dressing. Anesthetics and analgesics may include lidocaine, benzocaine, procaine, aloe, menthol, paracetamol, non-steroidal anti-inflammatory drugs and opioid drugs. Antibiotics, anesthetics, and analgesics may be present in the burn dressing in amounts of less than about 2.0%.

Preferably, the spray-on burn dressing has a pH of about 4.0 to about 7.5. In one embodiment the spray-on burn dressing has a pH of about 4.0 to about 7.0. In one embodiment the spray-on burn dressing has a pH similar to that of skin from about 5.5 to about 6.0. In one embodiment the spray-on burn dressing has a pH higher than that of skin from about 6.0 to about 7.5.

In one embodiment heparan sulfate is included in the burn dressing as an anticoagulant and promoter of wound healing. In one embodiment other glycosaminoglycans including heparin, dermatan sulfate, keratin sulfate, chondroitin-4 and chondroitin-6-sulfate, and hyaluronic acid can be added to accelerate wound healing.

While the present inventions have been illustrated and described in many embodiments of varying scope, it will at once be apparent to those skilled in the art that variations may be made within the spirit and scope of the inventions. Accordingly, it is intended that the scope of the inventions set forth in the appended claims not be limited by any specific wording in the foregoing description, except as expressly provided.

What is claimed is:

1. A spray-on burn dressing comprising: a film-forming polymer and aluminum oxide particles.

2. A dressing of claim 1 further comprising a solvent.

3. A dressing of claim 2 further comprising heparan sulfate

4. A dressing of claim 1 wherein said film-forming polymer is methyl vinyl ether/maleic acid copolymer, wherein said aluminum oxide particles have a diameters between about 5 and about 100 nanometers, and further including isopropanol and water.

5. A method of treating a burn comprising: applying a thermally conductive composition to a burn, said composition comprising a film-forming polymer with aluminum oxide particles.

6. A method of claim 5 wherein the composition is applied via aerosol spray.

7. A burn dressing composition comprising: about 1% to about 20% of methyl vinyl ether/maleic acid copolymer, about 10% to about 90% isopropanol, about 10% to about 40% water, and about 10% to about 30% aluminum oxide microparticles.

8. A burn dressing of claim 7 comprising about 2% of methyl vinyl ether/maleic acid copolymer, about 50% isopropanol, about 28% water, and about 20% aluminum oxide microparticles.

9. A burn dressing of claim 8 where said aluminum oxide microparticles have a mean size of about 10 micrometers.

10. A method of healing burnt skin, the method comprising: applying a dressing to the burnt skin, wherein the dressing is made in accordance with any of claims 1-9; and improving heat dissipation from the burnt skin.
